(19) United States
(12) Patent Application Publication
(10) Pub. No.: US 2019/0228205 A1
(43) Pub. Date: Jul. 25, 2019

(54) SKINPRINT ANALYSIS METHOD AND APPARATUS

(71) Applicant: INTELLIGENT FINGERPRINTING LIMITED, Cambridge (Cambridgeshire) (GB)

(72) Inventors: Benjamin Gordon, Cambridge (Cambridgeshire) (GB); Paul Wilson, Cambridge (Cambridgeshire) (GB)

(73) Assignee: INTELLIGENT FINGERPRINTING LIMITED, Cambridge (Cambridgeshire) (GB)

(21) Appl. No.: 16/324,793

(22) PCT Filed: Aug. 10, 2017
(86) PCT No.: PCT/GB2017/052365

§ 371 Date: Feb. 11, 2019

(30) Foreign Application Priority Data
Aug. 11, 2016 (GB) 1613819.0

(51) Int. Cl. G06K 9/00; G06K 9/20; G06T 7/00

(52) U.S. Cl. CPC G06K 9/0004; G06K 9/2036; G06T 2207/30196; G06K 9/00087; G06T 2207/30168; G06T 7/0002

(57) ABSTRACT

A method of determining presence of a skinprint uses an apparatus comprising: a primary electromagnetic radiation source; an electromagnetic radiation detector; and a translucent waveguide comprising a first surface providing a waveguide interface coincident with a skinprint receiving region. The method comprises: transmitting primary electromagnetic radiation from the primary electromagnetic radiation source towards the waveguide interface at an angle of incidence relative to and on a first side of a normal line that is perpendicular to the waveguide interface, such that: (a) where the waveguide interface interfaces directly with ambient, the primary electromagnetic radiation incident on the waveguide interface reflects in the waveguide interface at an angle of reflection relative to and on a second side of the normal line opposite the first side; and (b) where a skinprint is present on the skinprint receiving region such that the waveguide interface interfaces with the skinprint and the skinprint interfaces with ambient, at least a portion of the primary electromagnetic radiation incident on the waveguide interface is transmitted through the waveguide interface into the skinprint. The method further comprises using the electromagnetic radiation detector to determine an amount of primary electromagnetic radiation transmitted through the waveguide interface and/or reflected by the waveguide interface. Also disclosed is an apparatus for carrying out the method.

### TECHNICAL FIELD

The disclosure relates to analysis of a skinprint, such as a fingerprint. The analysis may confirm the presence of a skinprint and may also provide an indication of the quality and/or quantity of the skinprint. The analysis may also confirm the identity of the skinprint.

### BACKGROUND

Skinprints comprise eccrine sweat and may contain other constituents that may form a target for a diagnostic test. The applicant has developed a range of techniques for detecting the presence of one or more analytes in skinprints.

Characteristics of skinprints may vary substantially, for example in terms of area of skinprint and quantity of print substances present in the skinprint. These and other characteristics may contribute to a measure of skinprint quality/quantity. Skinprint quality/quantity may considerably affect the ease or otherwise of analysing the skinprint both for identification purposes and also for the purpose of detecting one or more analytes in the skinprint, especially when seeking quantitative analysis of one or more analytes.

By way of example, a good quality skinprint, on which the detection of one or more analytes may be most straightforward, may be one that is provided by a user depositing a firm impression of unwashed skin on a surface. By contrast, a reduced quality skinprint may be provided by a user who has recently washed the relevant area of skin and/or who provides only a minimal force when depositing the impression on a surface.

In some circumstances, for example, it may be that a user deliberately washes their hands in anticipation of being asked to provide a fingerprint and also uses only a minimal force when leaving the fingerprint.

It may be beneficial to analyse a skinprint for a measure of quality/quantity for a number of reasons. Such reasons may include (but are not necessarily limited to): confirming that a skinprint is present at all; confirming (where a skinprint is present) that the skinprint is of sufficient quality/quantity to facilitate a meaningful analysis of analytes; determining (where a skinprint is present) whether quality/quantity is sufficient for a quantitative analysis of analytes to be performed.

In addition, providing a straightforward quality check may avoid cost and inefficiency associated with analyte testing on a sample that is sub-standard or even absent altogether.

A quality check may be undertaken before or after collecting a skinprint for diagnostic analysis as well as being an integral part of a diagnostic analysis of materials such as metabolites in the skinprint.

It is known to use a quartz crystal microbalance to measure small mass increments. This technique does not lend itself well to robust in-the-field determination of fingerprint or skinprint mass measurement. Furthermore, while skinprint quantity may correspond with mass, the relationship between skinprint quality and mass may be more complex. The applicant has identified a need for a rugged, reliable system with low cost consumables for the purpose of analysing skinprint quality/quantity.

### SUMMARY OF THE DISCLOSURE

Against this background, there is provided a method of determining presence of a skinprint using an apparatus comprising: a primary electromagnetic radiation source; an electromagnetic radiation detector; and a translucent waveguide comprising a first surface providing a waveguide interface coincident with a skinprint receiving region;
the method comprising:
transmitting primary electromagnetic radiation from the primary electromagnetic radiation source towards the waveguide interface at an angle of incidence relative to and on a first side of a normal line that is perpendicular to the waveguide interface, such that:
(a) where the waveguide interface interfaces directly with ambient, the primary electromagnetic radiation incident on the waveguide interface reflects in the waveguide interface at an angle of reflection relative to and on a second side of the normal line opposite the first side; and
(b) where a skinprint is present on the skinprint receiving region such that the waveguide interface interfaces with the skinprint and the skinprint interfaces with ambient, at least a portion of the primary electromagnetic radiation incident on the waveguide interface is transmitted through the waveguide interface into the skinprint; and
using the electromagnetic radiation detector to determine an amount of primary electromagnetic radiation transmitted through the waveguide interface and/or reflected by the waveguide interface.

In this way, a skinprint may be used to couple electromagnetic radiation into or out of a translucent waveguide. The extent of the coupled electromagnetic radiation is detected or at least inferred and thereby provides an indication of the quality/quantity of the skinprint.

In a further aspect of the disclosure, there is provided an apparatus for determining presence of a skinprint, the apparatus comprising:
a translucent waveguide comprising a first surface providing a waveguide interface coincident with a skinprint receiving region;
a primary electromagnetic radiation source coupled to the translucent waveguide such that primary electromagnetic radiation emitted by the primary electromagnetic radiation source reaches the waveguide interface at an angle of incidence relative to and on a first side of a normal line that is perpendicular to the waveguide interface so as to enable reflection of the primary electromagnetic radiation at the waveguide interface where the waveguide interfaces with ambient and so as to enable transmission of primary electromagnetic radiation through the waveguide interface where the waveguide interfaces with a skinprint;
an electromagnetic radiation detector configured to produce a detector signal indicative of either:
   (a) a first portion of the primary electromagnetic radiation that undergoes total internal reflection at the waveguide interface; or
   (b) a second portion of the primary electromagnetic radiation that undergoes transmission through the waveguide interface; and
a processor configured to process the detector signal and use it to output a result indicative of an extent to which primary electromagnetic radiation behaviour is impacted by presence of a skinprint on the skinprint receiving region.

Embodiments of the disclosure will now be described, by way of example only, with reference to the accompanying drawing in which:

### BRIEF DESCRIPTION OF THE DRAWINGS

FIG. 1a provides a schematic representation of a first embodiment of the disclosure showing behaviour of electromagnetic radiation in the event that a skinprint is present;

FIG. 1b provides a schematic representation of the first embodiment of the disclosure showing behaviour of electromagnetic radiation in the event that no skinprint is present;

FIG. 2a provides a schematic representation of a second embodiment of the disclosure showing behaviour of electromagnetic radiation in the event that a skinprint is present;

FIG. 2b provides a schematic representation of the second embodiment of the disclosure showing behaviour of electromagnetic radiation in the event that no skinprint is present;

FIG. 3a provides a schematic representation of a third embodiment of the disclosure showing behaviour of electromagnetic radiation in the event that a skinprint is present;

FIG. 3b provides a schematic representation of the third embodiment of the disclosure showing behaviour of electromagnetic radiation in the event that no skinprint is present;

FIG. 4a provides a schematic representation of a fourth embodiment of the disclosure showing behaviour of electromagnetic radiation in the event that a skinprint is present;

FIG. 4b provides a schematic representation of the fourth embodiment of the disclosure showing behaviour of electromagnetic radiation in the event that no skinprint is present;

FIG. 5a provides a schematic representation of a fifth embodiment of the disclosure showing behaviour of electromagnetic radiation in the event that a skinprint is present;

FIG. 5b provides a schematic representation of the fifth embodiment of the disclosure showing behaviour of electromagnetic radiation in the event that no skinprint is present;

FIG. 6a provides a schematic representation of a sixth embodiment of the disclosure showing behaviour of electromagnetic radiation in the event that a skinprint is present;

FIG. 6b provides a schematic representation of the sixth embodiment of the disclosure showing behaviour of electromagnetic radiation in the event that no skinprint is present;

FIG. 7a provides a schematic representation of a seventh embodiment of the disclosure showing behaviour of electromagnetic radiation in the event that a skinprint is present;

FIG. 7b provides a schematic representation of the seventh embodiment of the disclosure showing behaviour of electromagnetic radiation in the event that no skinprint is present;

FIG. 8a provides a schematic representation of an eighth embodiment of the disclosure showing behaviour of electromagnetic radiation in the event that a skinprint is present; and

FIG. 8b provides a schematic representation of the eighth embodiment of the disclosure showing behaviour of electromagnetic radiation in the event that no skinprint is present.

### DETAILED DESCRIPTION

The disclosure relates to a method and apparatus for determining presence of a skinprint 30. A wide range of alternative implementations is envisaged. The following detailed description relates to a subset of embodiments that fall within the scope of the appended claims.

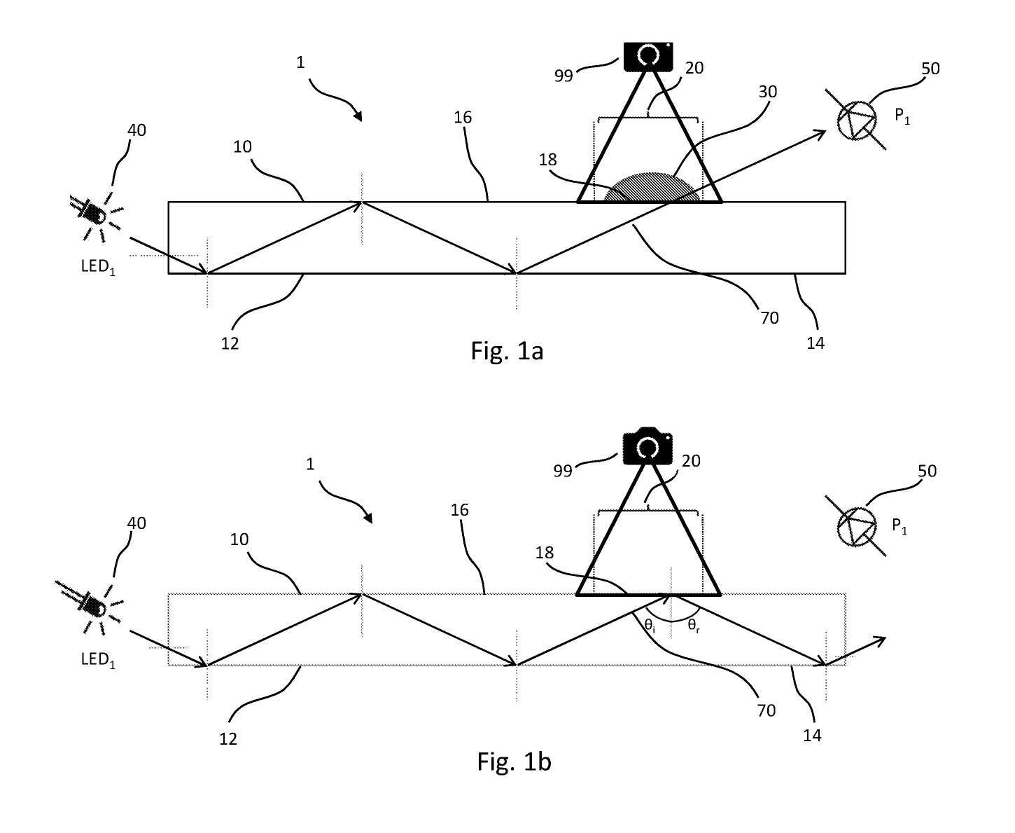

FIGS. 1a and 1b show a schematic representation of a first embodiment 1 of the disclosure. FIG. 1a shows behaviour of electromagnetic radiation in the first embodiment 1 where a skinprint 30 is present while FIG. 1b shows behaviour of electromagnetic radiation in the first embodiment where no skinprint is present. The first embodiment 1 comprises an LED 40, a photodiode 50 and a translucent waveguide 10 between the LED 40 and the photodiode 50 configured to output a photodiode signal indicative of electromagnetic radiation detected by the photodiode 50.

The translucent waveguide 10 comprises a first end 12 and a second end 14. The LED 40 is optically coupled to the translucent waveguide 10 towards the first end 12.

The second end 14 comprises a fingerprint receiving region 20 on a first surface 16 of the translucent waveguide 10. The fingerprint receiving region 20 may be identified on the first surface 16 by virtue of one or more visible indications on or surrounding the fingerprint receiving region 20. Alternatively, the fingerprint receiving region 20 may be identified by a window bounded by a frame that obscures parts of the first surface 16 that do not form part of the fingerprint receiving region 20. The fingerprint receiving region 20 may be identified by other means.

A surface of the translucent waveguide 10 in the vicinity of the fingerprint receiving region 20 may serve as a waveguide interface 18 through which electromagnetic radiation may be transmitted or in which electromagnetic radiation may be reflected, dependent upon circumstances. The waveguide interface 18 may or may not be different in surface properties when compared to a surface of the translucent waveguide 10 that surrounds the waveguide interface 18.

The photodiode 50 is located so as to detect electromagnetic radiation that is transmitted out of the translucent waveguide 10 via the waveguide interface 18.

The LED 40 is optically coupled to the translucent waveguide 10 towards the first end 12 such that electromagnetic radiation 70 emitted by the LED 40 enters into the translucent waveguide 10 at an angle such that the electromagnetic radiation 70 is retained within the translucent waveguide by total internal reflection. Optical coupling of the LED 40 to the translucent waveguide 10 may take any appropriate form. At the point of entry of the electromagnetic radiation 70 into the translucent waveguide 10, some refraction of the electromagnetic radiation 70 may take place. (For the sake of clarity, this refraction is not shown in the Figures.) In particular, electromagnetic radiation 70 that is incident upon an end surface of the translucent waveguide 10 at an angle of incidence is transmitted into the translucent waveguide 10 with a small change in direction away from a normal line (which is shown in the Figure) that is perpendicular to the surface through which the electromagnetic radiation 70 enters the translucent waveguide 10. The extent of the refraction that takes place depends upon the ratio between the index of refraction of the translucent waveguide 10 and the index of refraction of the material through which the electromagnetic radiation 70 travels immediately prior to reaching the point of entry. Where the material immediately prior to the electromagnetic radiation 70 reaching the point of entry is ambient air, the ratio is likely to be higher (and so the extent of the refraction is likely to be greater) than if the material immediately prior to the electromagnetic radiation 70 reaching the point of entry is, for example, a translucent encapsulation material of an LED package. Accordingly, the nature and extent of any refraction will depend upon how the electromagnetic radiation 70 is coupled from the electromagnetic radiation source 40 into the translucent waveguide 10.

Subsequently, as the electromagnetic radiation 70 travelling within the translucent waveguide 10 reaches the edges of the translucent waveguide 10, it arrives at an angle of incidence that is such as to cause the electromagnetic radiation 70 to reflect at the perimeter of the translucent waveguide 10 as a result of total internal reflection rather than to be transmitted out of the translucent waveguide 10. This pattern of total internal reflection is reproduced along the translucent waveguide 10 and by this mechanism the electromagnetic radiation 70 propagates along and within the translucent waveguide 10.

While in FIG. 1a a skinprint 30 is shown in situ on the skinprint receiving region 20, in FIG. 1b no skinprint is present on the skinprint receiving region 20. A comparison between FIGS. 1a and 1b illustrates how behaviour of electromagnetic radiation 70 is influenced by the presence or absence of a skinprint 30 on the skinprint receiving region.

In the case that no skinprint is present on the skinprint receiving region, as is evident from FIG. 1b, a further total internal reflection occurs at the location of the skinprint receiving region 20 such that the electromagnetic radiation 70 continues to propagate along the translucent waveguide 10. When the electromagnetic radiation 70 reaches the end of the translucent waveguide 10, it arrives at an angle such that it passes through the end of the translucent waveguide 10, albeit undergoing some refraction (again for the sake of clarity not shown in FIG. 1b) and thereby exits the translucent waveguide 10.

By contrast, as can be seen from FIG. 1a, in the case that a skinprint 30 is present on the skinprint receiving region 20, at least a portion of the electromagnetic radiation 70 that arrives at the skinprint receiving region 20 is transmitted out of the translucent waveguide 10 at the waveguide interface 18 by virtue of the presence of the skinprint 30. This is because the waveguide interface 18 is (at least partially) covered by residue of the constituents of the skinprint, hereafter for brevity referred to simply as the skinprint 30. Therefore, instead of the waveguide interface 18 interfacing directly with ambient conditions (such as ambient air) wherein the difference in refractive indices between the translucent waveguide 10 and ambient would be such as to result in total internal reflection, the waveguide interface 18 interfaces directly with the skinprint 30. The ratio of refractive indices between that for the translucent waveguide 10 and that for the skinprint 30 is such that at least some of the electromagnetic radiation 70 is transmitted through the waveguide interface 18 and into the skinprint. When the electromagnetic radiation 70 reaches the surface of the skinprint (opposite the translucent waveguide 10) a combination of the ratio of refractive indices between that for the skinprint 30 and that for the ambient together with the angle of incidence of the electromagnetic radiation 70 at the interface results in at least some of the electromagnetic radiation 70 being transmitted out of the skinprint 30.

In the embodiment of FIGS. 1a and 1b, electromagnetic radiation 70 that is transmitted via the waveguide interface 18 and out of the skinprint 30 is received at the photodiode 50. In very general terms, the greater the (influence of) the skinprint, the more electromagnetic radiation 70 is received by the photodiode 50. Accordingly, there is a relationship between the quality and/or extent of skinprint 30 on the skinprint receiving region 20 and the amount of electromagnetic radiation 70 detected by the photodiode 50. Where no skinprint is present, little or no electromagnetic radiation 70 will be detected by the photodiode 50 because it remains within the translucent waveguide 10. Where a well-defined, strong skinprint is present, a significant proportion of the electromagnetic radiation 70 will be coupled out of the waveguide interface and will reach the photodiode 50.

It should be noted that FIGS. 1a and 1b (as well as the corresponding Figures relating to other embodiments) are highly schematic. As the skilled person would readily understand, the analysis is not binary. That is to say, it is not the case that in the event of a skinprint 30 being present all electromagnetic radiation 70 will transmit out of the translucent waveguide 10 via the waveguide interface 18. Similarly, it is not the case that in the event of no skinprint is present, no electromagnetic radiation 70 will transmit out of the translucent waveguide via the waveguide interface 18. In reality, some electromagnetic radiation 70 will transmit out of the translucent waveguide when no skinprint 30 is present. Conversely, when a skinprint 30 is present some electromagnetic radiation 70 will remain in the translucent waveguide.

Furthermore, it should be noted that the electromagnetic radiation 70 will not all travel in exactly the directions indicated by the arrows in FIGS. 1a and 1b. In short, FIGS. 1a and 1b are schematic and are intended to illustrate the principles.

In the Figures, the schematic representation of a skinprint 30 (where present) is such as to suggest that it is manifested as a single dome-shaped form on the skinprint receiving region 20. It is emphasised that this representation is highly schematic. Again as the skilled person readily appreciates, the form of skinprints varies significantly depending upon many factors including the amount of eccrine sweat on the surface of the skin when printed and the force with which a user places the skin against the skinprint receiving region 20 when providing a skinprint. In reality, the skinprint is likely to comprise a number of peaks and troughs, all of which may influence the behaviour of electromagnetic radiation incident upon it in a variety of ways.

As can be seen from FIGS. 1a and 1b, the first embodiment may further comprise optical imaging capability, as illustrated schematically by a camera icon 99. The optical imaging capability may be employed to provide an optical image of the skinprint that might be compared with a database of skinprint images, so as to confirm identity of a skinprint. The optical image functionality is equally applicable to any of the other embodiments disclosed herein but, for the sake of clarity, it is not illustrated other than in FIGS. 1a and 1b.

The applicant has developed various techniques for chemical analysis of skinprints. In order to determine that the chemical analysis is feasible for a given skinprint, it is helpful to have an indication that there is sufficient material present in a skinprint in order to apply a particular chemical test and, in particular, to quantify results of the chemical analysis relative to a mass or volume of the skinprint under test. The techniques described herein provide an indication of the amount of skinprint (hereinafter referred to skinprint quality) that has been deposited on the skinprint receiving region. Where skinprint quality is high, the influence of the skinprint on the behaviour of electromagnetic radiation will be higher than when the skinprint quality is low which will in turn be higher than when there is no skinprint present.

While the techniques described herein may be useful for providing a binary output that simply indicates whether a skinprint is of sufficient quality for a chemical analysis to be performed (by exceeding a fingerprint quality threshold), for skinprints that meet this threshold it may also be desirable to provide a more granular quantitative output. This may in turn be used to provide an indication of a quantum of a particular chemical constituent that may be expected. For example, a high quality skinprint may be expected to contain more of a particular chemical than a lower (but still adequate) quality skinprint. Accordingly, if the subsequent chemical analysis is intended not only to detect for presence of a chemical but also for an indication of concentration of that chemical, a quantitative analysis of the quality of the skinprint may be used in this determination.

The apparatus of the first embodiment may comprise controller circuitry configured to receive the photodiode signal and process that signal in order to determine whether a skinprint quality threshold is met. It may also be configured to determine a metric for quality of the skinprint. The controller may, for example, be configured to receive a first (reference) photodiode signal prior to a user providing a skinprint on the skinprint receiving region and to receive a second photodiode signal once a skinprint has been provided on the skinprint receiving region and to compare the first and second signals. It may also be configured to make a comparison with a reference value indicative of a theoretical maximum that would be achieved in the event of a maximum quality skinprint. By appropriate processing of the first and second signals relative to the reference value a skinprint quality value may be calculated for a particular skinprint and output via a display or as data transmitted for onward processing and/or storage. Alternatively or in addition, it may be that the result is simply compared to a threshold to determine if it meets a predetermined criterion or criteria for a meaningful analysis and a simple binary output may be provided such as a red/green indication (in the manner of traffic lights).

The skilled person will recognise that there are a large number of options applicable to the first embodiment of the disclosure for processing data such as photodiode signals as well as data relating to the electromagnetic radiation emitted by the LED in order to calculate a skinprint quality value. Moreover, in some of the subsequent embodiments that include additional features and functionality, there may be further inputs for calculation of the skinprint quality value. In some or all of these embodiments, there may be a calibration technique as a precursor to performing the analysis that results in the skinprint quality value.

It may be that where a quantitative skinprint quality value is provided, this provides an input to an algorithm related to chemical analysis of the skinprint thereby providing a reference for an amount of chemical that might be expected in a skinprint of that particular quality value. In addition or instead it may be that the skinprint quality value is output to a chemical analysis process simply to confirm that the skinprint is of sufficient quality/quantity to be appropriate for chemical analysis. In this way, it may be possible to avoid the time and expense associated with performing chemical analysis on a skinprint in which there is no confidence that a meaningful chemical analysis can be performed because the quality/quantity of the skinprint is insufficient.

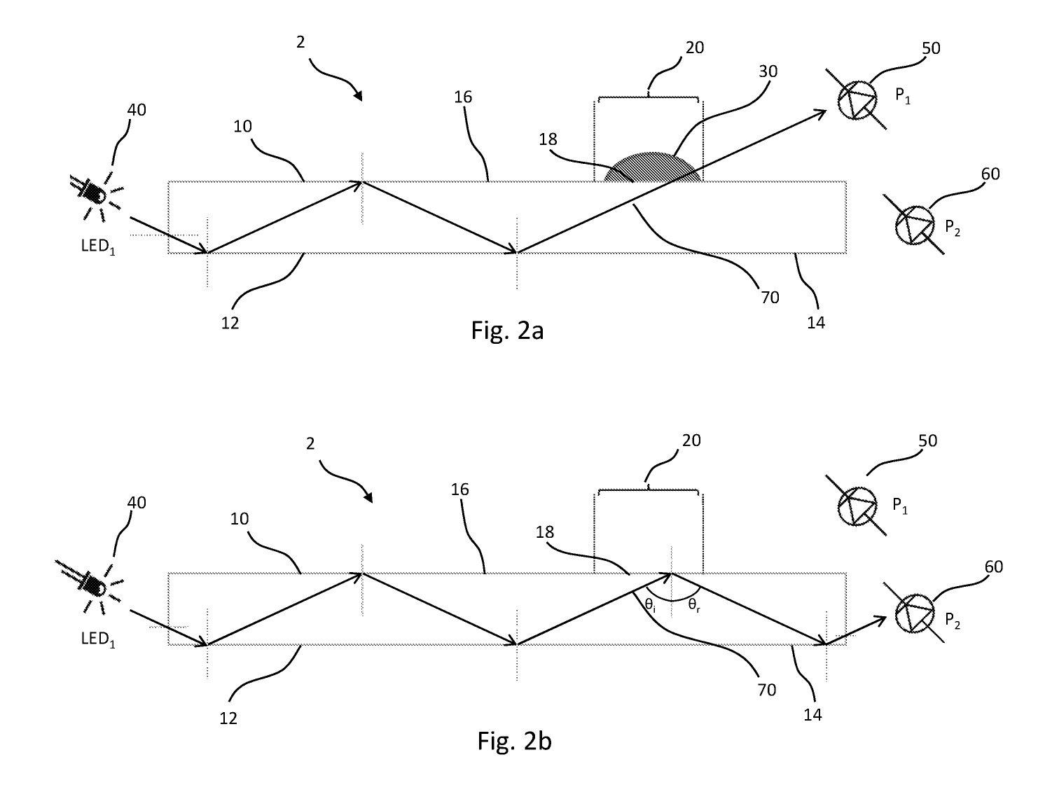

FIGS. 2a and 2b show a second embodiment 2 of the disclosure. The second embodiment 2 of the disclosure differs from the first embodiment in that a second photodiode 60 is provided in addition to the first photodiode 50. The second photodiode 60 is intended to detect electromagnetic radiation 70 that is not transmitted through the waveguide interface and is instead propagated by total internal reflection throughout the translucent waveguide 10. By providing two photodiodes and obtaining a signal from each indicative of an amount of electromagnetic radiation detected by each, the signals from each of the first and second photodiodes 50, 60 can be compared as part of a calculation to determine a skinprint quality value.

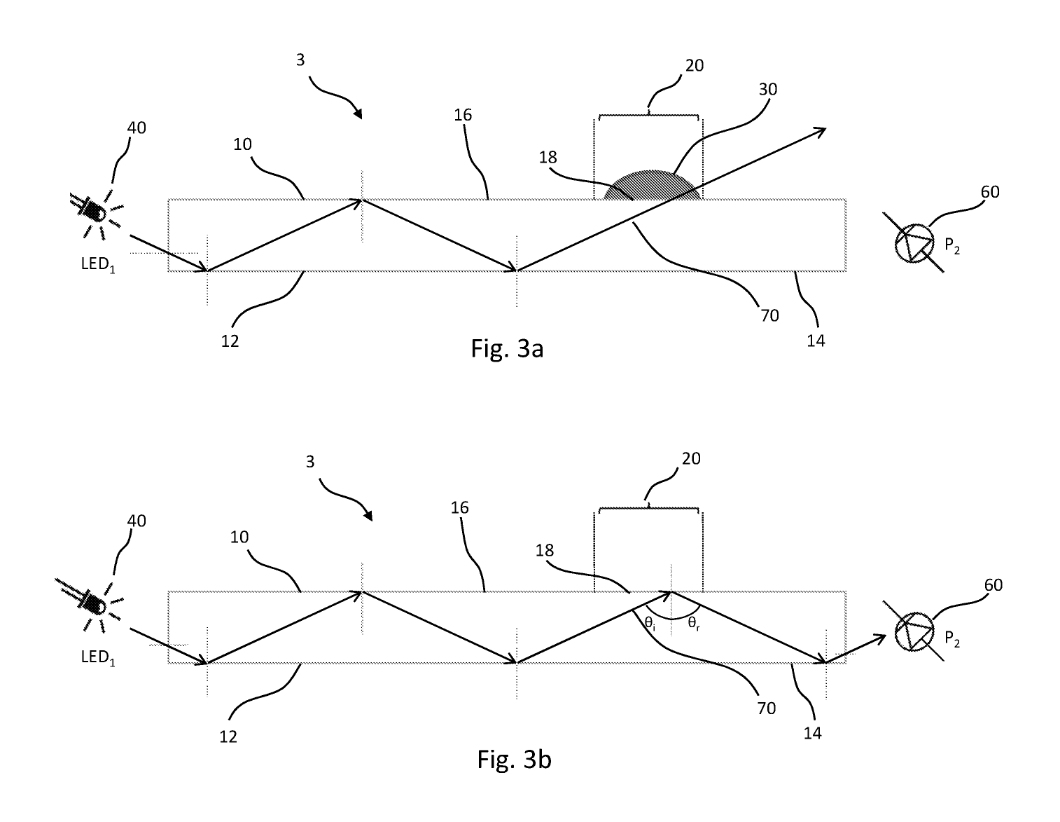

FIGS. 3a and 3b show a third embodiment 3 of the disclosure. The third embodiment 3 of the disclosure differs from the first and second embodiments 1, 2 in that only the second photodiode 60 (and not the first photodiode) is provided. In this way, the photodiode 60 only detects electromagnetic radiation 70 that is not transmitted through the waveguide interface and is instead propagated by total internal reflection throughout the translucent waveguide 10.

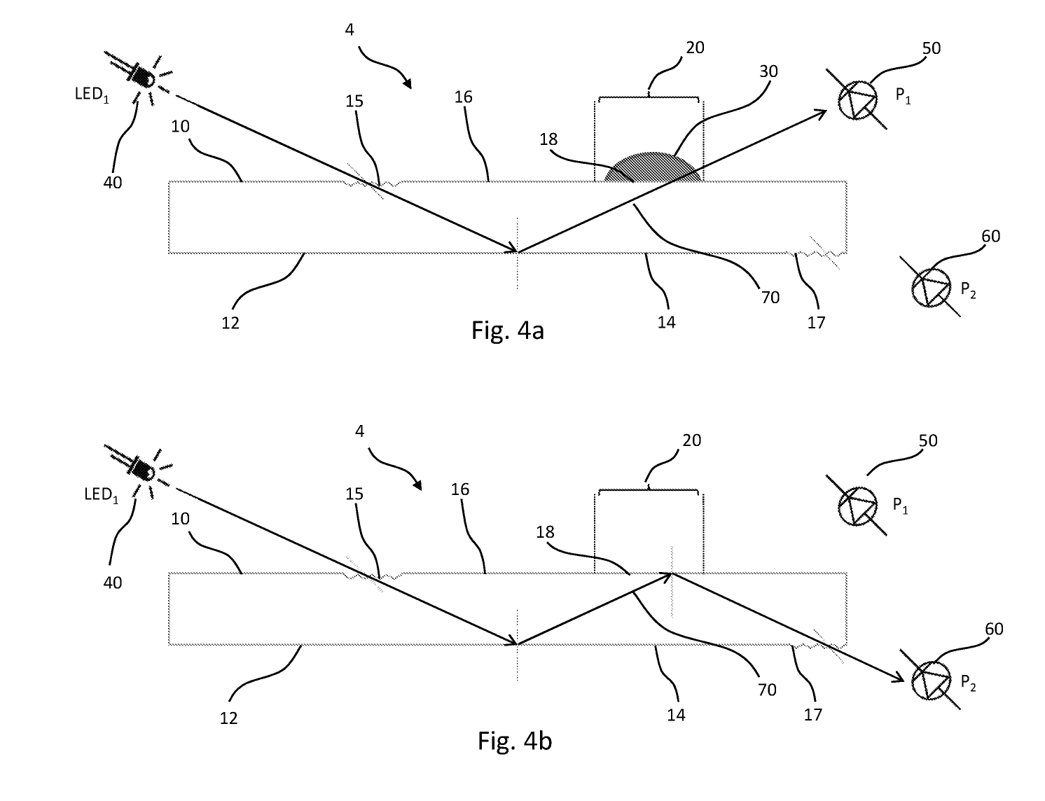

FIGS. 4a and 4b show a fourth embodiment 4 of the disclosure. The fourth embodiment 4 of the disclosure differs from the second embodiment 2 in that electromagnetic radiation 70 is transmitted (coupled) into the translucent waveguide 10 via a first grating coupler 15 and in that electromagnetic radiation 70 that is not transmitted out of the waveguide interface 18 and continues to propagate through the translucent waveguide 10 by total internal reflection is transmitted (coupled) out of the translucent waveguide 10 via a second grating coupler 17. The first grating coupler 15 may comprise a roughened portion of a surface of the translucent waveguide 10 through which electromagnetic radiation may pass into the translucent waveguide 10. This may provide flexibility regarding location of the LED 40 relative to the translucent waveguide 10. This may be particularly appropriate when providing the apparatus in a compact portable package. The second grating coupler 17 may comprise a roughened portion of a surface of the translucent waveguide 10 through which electromagnetic radiation may pass out of the translucent waveguide 10. This may provide flexibility regarding location of the second photodiode 60 relative to the translucent waveguide 10. Again, this may be particularly appropriate when providing the apparatus in a compact portable package.

As the skilled person would readily appreciate, alternative embodiments (not illustrated) may involve only one of the two grating couplers 15, 17. For example, an alternative embodiment may include a first grating coupler 15 in the absence of a second grating coupler 17. In such an embodiment electromagnetic radiation 70 that is not transmitted out of the waveguide interface 18 and continues to propagate through the translucent waveguide 10 by total internal reflection may be transmitted (coupled) out of the translucent waveguide 10 in the same manner as in the second and third embodiments 2, 3. Similarly, a further alternative embodiment may include a second grating coupler 17 in the absence of a first grating coupler 15. In such an embodiment, electromagnetic radiation 70 may be coupled into the translucent waveguide 10 in the same manner as for the first, second and third embodiments 1, 2, 3.

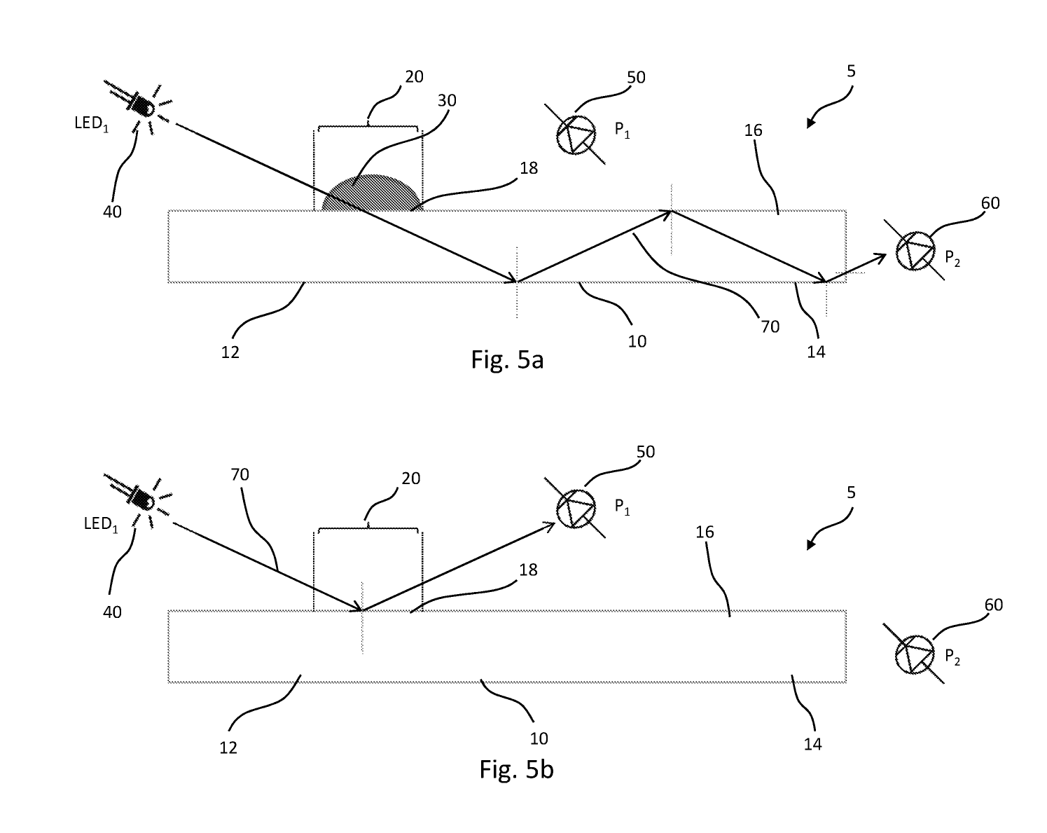

FIGS. 5a and 5b show a fifth embodiment 5 of the disclosure. The fifth embodiment 5 differs from the first to fourth embodiments 1, 2, 3, 4 in that the direction of potential transmission through the waveguide interface 18 (in the presence of a skinprint) is into the translucent waveguide 10 rather than out of the translucent waveguide 10. Accordingly, the electromagnetic radiation source 40 is located such that electromagnetic radiation 70 reaches the waveguide interface 18 from the exterior of the translucent waveguide 10 towards the first end 12 of the translucent waveguide 10. In addition, the fingerprint receiving region 20 is located on the first surface 16 of the translucent waveguide 10 also towards the first end 12 of the translucent waveguide 10. In the event that a skinprint is present, electromagnetic radiation 70 is transmitted through the waveguide interface 18 and into the translucent waveguide 10 for onward propagation towards the second end 14 of the translucent waveguide 14 through total internal reflection as shown schematically in FIG. 5a. In the event that no skinprint is present, electromagnetic radiation simply reflects off the waveguide interface 18 and thereby never enters the translucent waveguide 10 as shown in FIG. 5b. In the illustration, electromagnetic radiation that is reflected in the waveguide interface 18 is detectable by a first photodiode 50 and electromagnetic radiation that is transmitted through the waveguide interface 18 via a skinprint is detectable by a second photodiode 60. However, in common with the differences between the first, second and third embodiments 1, 2, 3, it may be appropriate to have only one rather than both of the photodiodes.

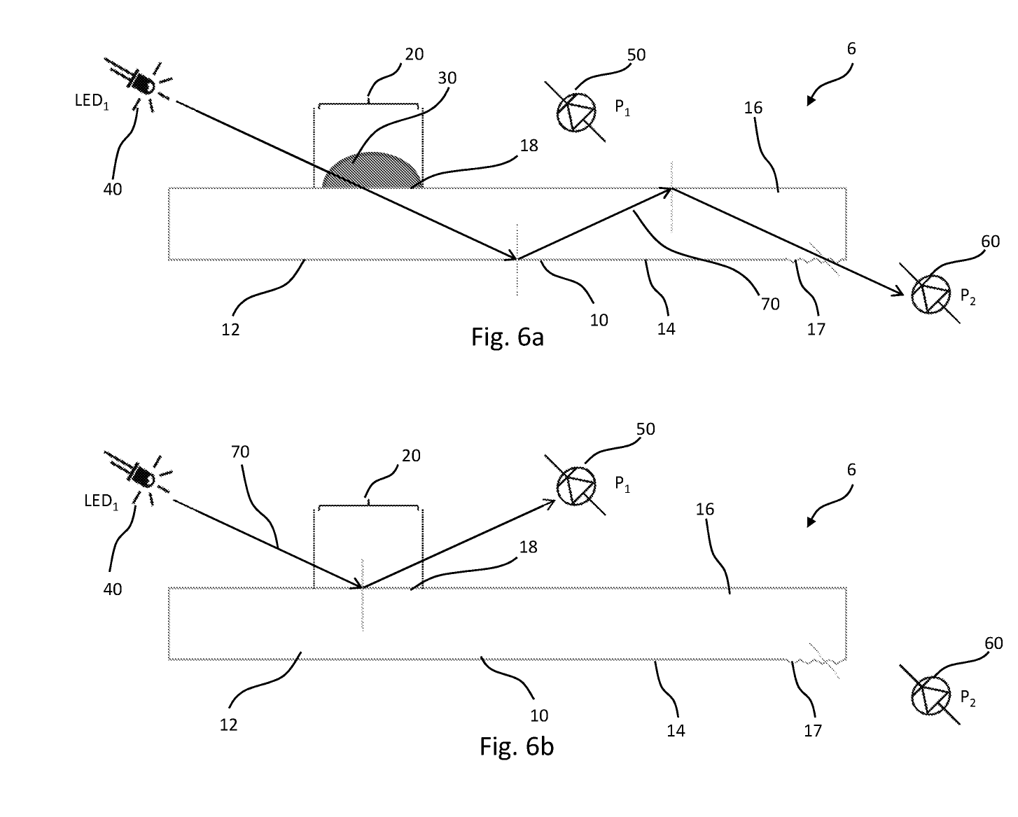

FIGS. 6a and 6b show a sixth embodiment 6 of the disclosure. The sixth embodiment 6 differs from the fifth embodiment 5 in that electromagnetic radiation 70 that is transmitted through the waveguide interface 18 via a skinprint 30 is transmitted out of the waveguide 10 via an output grating coupler 17, as described previously in relation to the fourth embodiment 4.

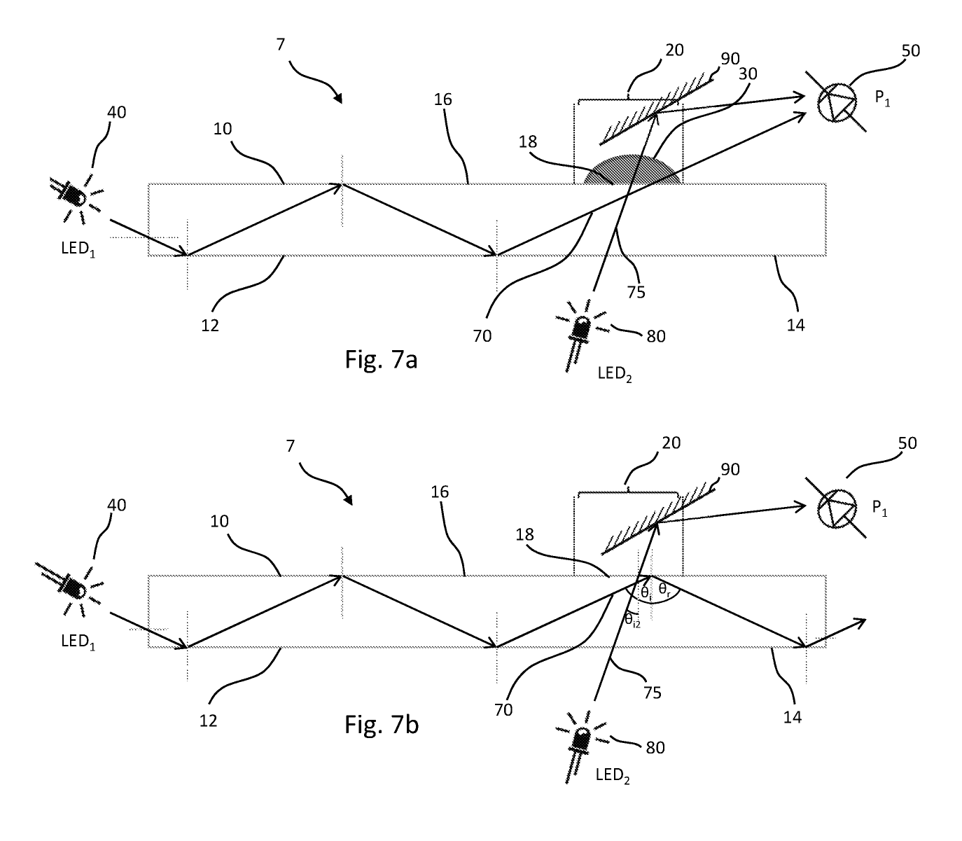

FIGS. 7a and 7b show a seventh embodiment 7 of the disclosure. The seventh embodiment 7 is similar to the first embodiment 1 and further comprises a secondary electromagnetic radiation source 80. The secondary electromagnetic radiation source 80 is located so that secondary radiation 75 emitted from the secondary electromagnetic radiation source 80 travels at an angle such that it transmits directly through the waveguide interface 18 whether or not a skinprint is present.

Accordingly, when no skinprint is present, only the secondary radiation 75 reaches the photodetector 50. This is because the primary electromagnetic radiation 70 from the primary electromagnetic radiation source 40 is reflected by the waveguide interface 18 rather than being transmitted through it. (A reflector 90 may be used to ensure that the secondary radiation, once out of the waveguide 10, is directed to the photodetector 50.)

When a skinprint 30 is present, primary radiation 70 from the primary radiation source 40 passes through the waveguide interface 18 such that both primary and secondary radiation 70, 75 reach the photodetector 50.

In one aspect of the seventh embodiment 7, one or both of the primary and secondary radiation sources 40, 80 may be pulsed. For example, if the primary radiation source 40 is constant and the secondary radiation source 80 is pulsed then the primary radiation 70 can be detected when the secondary radiation source 80 is off. A value for the secondary radiation 80 can be calculated by subtracting the measured primary radiation 70 from the measured combination of primary and secondary radiation when the secondary radiation source 80 is on.

If the primary and secondary radiation sources 40, 80 are of the same specification (e.g. in terms of brightness and spectrum) then they will both be affected by the material properties of the translucent waveguide 10 in the same way. Accordingly, it is possible by this technique to eliminate variations that arise from the use of different waveguides. This may be particularly appropriate where the waveguide 10 is a consumable product that is replaced with each test performed.

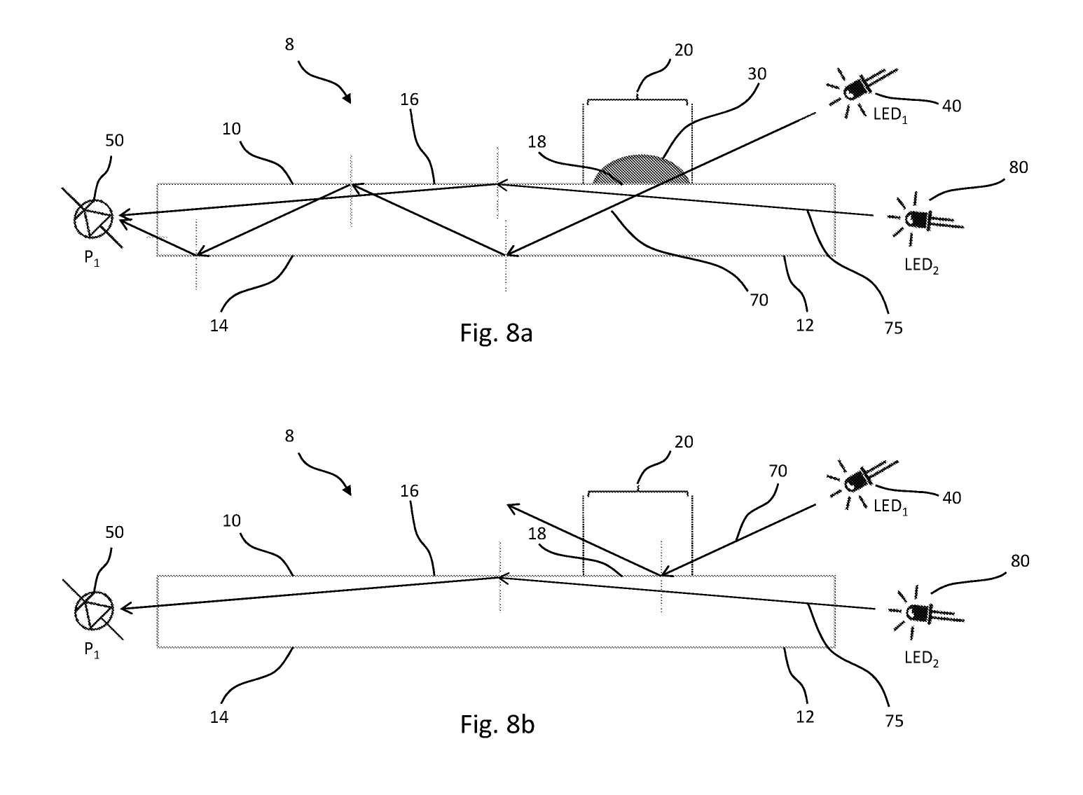

FIGS. 8a and 8b show an eighth embodiment of the disclosure. In common with the seventh embodiment 7, the eighth embodiment 8 comprises both primary and secondary electromagnetic radiation sources 40, 80. The primary and secondary electromagnetic radiation sources 40, 80 are both located towards a first end 12 of the translucent waveguide 10. The skinprint receiving region 20 is also located towards the first end 12 of the translucent waveguide 10. A photodetector 50 is located towards the second end 14 of the translucent waveguide 10.

In common with the fifth and sixth embodiments (and by contrast with the first, second, third, fourth and seventh embodiments), the direction of potential transmission through the waveguide interface 18 (in the presence of a skinprint) is into the translucent waveguide 10 rather than out of the translucent waveguide 10.

The primary electromagnetic radiation source 40 is located such that primary electromagnetic radiation 70 reaches the waveguide interface 18 from the exterior of the translucent waveguide 10 towards the first end 12 of the translucent waveguide 10. In addition, the fingerprint receiving region 20 is located on the first surface 16 of the translucent waveguide 10 also towards the first end 12 of the translucent waveguide 10. In the event that a skinprint is present, electromagnetic radiation 70 is transmitted through the waveguide interface 18 and into the translucent waveguide 10 for onward propagation towards the second end 14 of the translucent waveguide 14 through total internal reflection as shown schematically in FIG. 8a. In the event that no skinprint is present, as shown in FIG. 8b, primary electromagnetic radiation 70 from the primary electromagnetic radiation source 40 simply reflects off the waveguide interface 18 and does not enter the translucent waveguide 10.

The secondary electromagnetic radiation source 80 is located such that secondary radiation 75 is directed into the translucent waveguide 10 at an angle such that it propagates through the translucent waveguide 10 without opportunity for it to be coupled out of the translucent waveguide 10 until it reaches the second end 14 of the translucent waveguide in the region of the photodetector 50. This may be achieved by directing the secondary electromagnetic radiation 75 into the translucent waveguide 10 in a direction that is only marginally angled relative to the first surface 16 of the translucent waveguide 10 (or potentially substantially parallel to the first surface). In this way, the angle of travel of the secondary electromagnetic radiation 75 through the translucent waveguide 10 is such that neither the presence nor the absence of a skinprint 30 enables the radiation to be coupled out of the translucent waveguide 10, at least to any substantial degree.

Electromagnetic radiation (whether primary or secondary) that reaches the second end 14 of the translucent waveguide 10 is detected by the first photodiode 50. In the event that no skinprint 30 is present on the skinprint receiving region 20 (see FIG. 8b), primary electromagnetic radiation 70 will not be coupled into the translucent waveguide 10 via the waveguide interface 18 and therefore only secondary radiation 75 will arrive at the photodiode 50. By contrast (see FIG. 8a), in the event that a skinprint 30 is present on the skinprint receiving region 20, primary radiation 70 that is coupled into the translucent waveguide 10 via the waveguide interface 18 as a result of the presence of a skinprint 30, will arrive at the photodiode 50 in addition to secondary radiation 75.

As in the seventh embodiment, one or both of the primary and secondary radiation sources 40, 80 may be pulsed. For example, if the primary radiation source 40 is constant and the secondary radiation source 80 is pulsed then the primary radiation 70 can be detected in isolation when the secondary radiation source 80 is off. Where no skinprint 30 is present (such that no primary radiation would be expected to arrive at the photodiode 50) the photodiode would detect radiation only when the secondary radiation source 80 is on.

Alternatively, the secondary radiation source 80 may be constant and the primary radiation source 40 may be pulsed. In this way, where no skinprint is present there should be little difference between the radiation detected by the photodetector 50 regardless of the pulsed nature of the primary radiation 70 since the primary radiation 70 (when on) is not coupled into the translucent waveguide 10 and therefore does not reach the photodetector 50.

If the primary and secondary radiation sources 40, 80 are of the same specification (e.g. in terms of brightness and spectrum) then they will both be affected by the material properties of the translucent waveguide 10 in the same way. Accordingly, it is possible by this technique to eliminate variations that arise from the use of different waveguides. This may be particularly appropriate where the waveguide 10 is a consumable product that is replaced with each test performed.

In any embodiment involving both primary and secondary radiation, as an alternative to the pulsing strategy for separating primary and secondary radiation detected at the photodetector 50, it may be possible to use primary radiation having a different colour from that of the secondary radiation and use a colour sensitive photodetector to distinguish between the primary and secondary radiation. In short, any appropriate technique for distinguishing between primary and secondary radiation may be employed. Such techniques may include separation in the frequency domain, separation in the time domain, and separation in the colour domain. Whichever separation technique may be employed, the concept is to distinguish between primary radiation (main path) and secondary radiation (reference path).

The skilled person will appreciate that aspects of different embodiments described herein may be combined, including in ways not explicitly recited. For example, in the case of the seventh embodiment, it may be appropriate to use two photodiodes, in the manner of embodiments 2, 4, 5 and 6. Similarly, it may be appropriate to use a secondary electromagnetic radiation source in any of embodiments 1 to 6.

The skilled person will understand that refraction necessarily occurs when electromagnetic radiation passes between materials having different refractive indices (unless, of course, the difference of refractive indices is such as to result in total internal reflection). For the sake of clarity only, refraction is not shown in the schematic representations of FIGS. 1a to 8b.

The angle of incidence, θᵢ, at which the primary electromagnetic radiation 70 reaches the waveguide interface 18 is necessarily greater than the angle of incidence, θᵢ₂, at which the secondary electromagnetic radiation 75 reaches the waveguide interface 18. The exact values for θᵢ and θᵢ₂ will depend, among other things, on the refractive indices of the material used for the translucent waveguide 10 and the material (e.g. ambient air) on adjacent the waveguide interface 18 of the translucent waveguide 10.

Where total internal reflection of the primary electromagnetic radiation 70 having the angle of incidence, θᵢ, occurs at the waveguide interface 18 it reflects at an angle of reflection, θᵣ.

While the schematic Figures show the electromagnetic radiation taking only a single path, as the skilled person will readily appreciate, the path of the radiation will diverge. The single lines shown in the Figures are intended to represent the average path of the radiation and for clarity the divergence of radiation from the average path is not shown.

The primary and/or secondary electromagnetic radiation sources may be a source of visible spectrum radiation. The primary and/or secondary light source may be an LED, a filament bulb, a laser, a fluorescent bulb, or any other suitable source of electromagnetic radiation.

The primary and/or secondary electromagnetic radiation may be broad spectrum or narrow spectrum radiation. Potentially, it may be two non-contiguous ranges of narrow spectrum radiation. In some embodiments, the primary and secondary electromagnetic radiation may have the same properties (e.g. wavelength); in other embodiments the primary and secondary electromagnetic radiation may be selected to have different properties (e.g. wavelength).

While the specific embodiments employ one or more photodiodes as electromagnetic radiation detector(s), any appropriate electromagnetic radiation detector(s) may be used. Choice of electromagnetic radiation detector may be dependent, among other things, on the electromagnetic radiation source. Possible electromagnetic radiation detectors include: a photodiode; a phototransistor; a CCD sensor; and a light dependent resistor.

It may be appropriate to use a camera and/or a photomultiplier instead of or in addition to the electromagnetic radiation detector(s) shown in the specific embodiments. In particular, a camera may be used to provide an image of the electromagnetic radiation which may be compared to a database of such images for confirming the identity of a skinprint subject.

While the term skinprint is used throughout this specification, it will be appreciated that the most frequently used form of skinprint is currently the fingerprint (which includes the thumb-print). Nevertheless, other skinprints may be appropriate, such as (but not limited to) a hand-print, a toe-print, a footprint or an ear-print.

The translucent waveguide of any of the embodiments may be any translucent having appropriate properties of transmissivity of electromagnetic radiation of the appropriate wavelengths. The translucent waveguide may be transparent. It may be a glass slide or a plastic slide. An off the shelf slide may be particularly appropriate in embodiments where the translucent waveguide is intended to be a consumable item whereby a new translucent waveguide is employed for each test. If a plastic slide is employed, it may be produced by injection moulding and optionally it may be plasma treated to obtain desirable waveguide properties.

1. A method of determining presence of a skinprint using an apparatus comprising: a primary electromagnetic radiation source; an electromagnetic radiation detector; and a translucent waveguide comprising a first surface providing a waveguide interface coincident with a skinprint receiving region;
the method comprising:
transmitting primary electromagnetic radiation from the primary electromagnetic radiation source towards the waveguide interface at an angle of incidence relative to and on a first side of a normal line that is perpendicular to the waveguide interface, such that:
(a) where the waveguide interface interfaces directly with ambient, the primary electromagnetic radiation incident on the waveguide interface reflects in the waveguide interface at an angle of reflection relative to and on a second side of the normal line opposite the first side; and
(b) where a skinprint is present on the skinprint receiving region such that the waveguide interface interfaces with the skinprint and the skinprint interfaces with ambient, at least a portion of the primary electromagnetic radiation incident on the waveguide interface is transmitted through the waveguide interface into the skinprint; and
using the electromagnetic radiation detector to determine an amount of primary electromagnetic radiation transmitted through the waveguide interface and/or reflected by the waveguide interface.

2. The method of claim 1 wherein the portion of the primary electromagnetic radiation that is transmitted through the waveguide interface is transmitted in a direction such as to exit the translucent waveguide.

3. The method of claim 2 wherein the step of transmitting primary electromagnetic radiation from the primary electromagnetic radiation source towards the waveguide interface is preceded by transmitting the primary electromagnetic radiation into the translucent waveguide at an angle so as to cause the primary electromagnetic radiation to propagate through the translucent waveguide by total internal reflection towards the waveguide interface.

4. The method of claim 3 wherein the translucent waveguide comprises an input grating coupler and wherein the step of transmitting the primary electromagnetic radiation into the translucent waveguide comprises transmitting the primary electromagnetic radiation towards the input grating coupler so as to enter the translucent waveguide.

5. The method of claim 3 or claim 4 wherein the translucent waveguide comprises an output grating coupler and wherein primary electromagnetic radiation that propagates within the translucent waveguide without transmitting through the waveguide interface exits the translucent waveguide via the output grating.

6. The method of claim 1 wherein the portion of the primary electromagnetic radiation that is transmitted through the waveguide interface is transmitted in a direction such as to enter the translucent waveguide.

7. The method of claim 6 wherein the step of transmitting primary electromagnetic radiation from the primary electromagnetic radiation source towards the waveguide interface involves transmitting the primary electromagnetic radiation at an angle such that the portion of primary electromagnetic radiation that is transmitted through the waveguide interface propagates through the translucent waveguide by total internal reflection.

8. The method of claim 7 wherein the translucent waveguide comprises an output grating coupler and such that the portion of primary electromagnetic radiation that is transmitted through the waveguide interface and propagates through the translucent waveguide by total internal reflection exits the translucent waveguide via the output grating.

9. The method of any preceding claim wherein the step of using the electromagnetic radiation detector to determine the amount of electromagnetic radiation transmitted through the waveguide interface and/or reflected by the waveguide interface involves one or both of the following:
using the electromagnetic radiation detector to detect an amount of electromagnetic radiation that exits the translucent waveguide having passed through the waveguide interface;
using the electromagnetic radiation detector to detect an amount of electromagnetic radiation that exits the translucent waveguide without having passed through the waveguide interface.

10. The method of any preceding claim further comprising inserting the translucent waveguide into the apparatus prior to performing the steps of claim 1.

11. The method of any preceding claim comprising calculating a quality metric for the skinprint based at least in part on an amount of primary electromagnetic radiation detected by the electromagnetic radiation detector to provide a value indicative of strength and/or extent of a skinprint on the skinprint receiving region.

12. The method of claim 11 wherein the step of calculating the quality metric comprises: comparing a signal from the electromagnetic radiation detector to a reference value.

13. The method of claim 12 wherein the apparatus further comprises a secondary electromagnetic radiation source configured to provide secondary electromagnetic radiation and the method further comprises detecting the secondary electromagnetic radiation source to provide the reference value.

14. The method of claim 13 wherein the secondary electromagnetic radiation is transmitted into the translucent waveguide at an angle such that the secondary electromagnetic radiation is transmitted through the waveguide interface without undergoing total internal reflection at the waveguide interface.

15. The method of claim 11 or claim 12 and further wherein the step of calculating the quality comprises calculating a ratio of detected primary electromagnetic radiation to detected secondary electromagnetic radiation.

16. The method of claim 13 or any claim dependent upon claim 13 further comprising pulsing either or both of the first electromagnetic radiation source and the second electromagnetic radiation source.

17. The method of any preceding claim wherein the translucent waveguide comprises an integrated reference feature and wherein the method further comprises using the electromagnetic radiation detector to detect the integrated reference feature and produce an output indicative thereof.

18. The method of claim 17 wherein the skinprint receiving region comprises the integrated reference feature.

19. The method of claim 17 or claim 18 when dependent directly or indirectly upon claim 3 or on any claim dependent upon claim 11 wherein calculation of the quality metric comprises comparing the output indicative of the integrated reference feature to a control value.

20. The method of any preceding claim wherein the primary electromagnetic radiation source is configured to produce broad spectrum electromagnetic radiation and wherein the calculation unit is configured to compare a spectrum of the electromagnetic radiation detected by the electromagnetic radiation detector with a spectrum of the electromagnetic radiation of the electromagnetic radiation source.

21. The method of any preceding claim further comprising capturing an image of the skinprint receiving region:

22. The method of claim 21 when dependent directly or indirectly upon claims 11 and 21 wherein the step of calculating the quality metric involves analysing the captured image of the skinprint to determine influence of the skinprint on the reference feature.

23. The method of claim 20 or any claim dependent upon claim 20 and further comprising: identifying spectral differences at wavelengths indicative of the presence of one or more particular constituents of human sweat to provide an indication of their potential presence.

24. The method of any of claims 1 to 19 wherein the primary electromagnetic radiation is of a specific wavelength selected for its sensitivity to one or more constituents that may be present in a skinprint.

25. The method of any preceding claim wherein the skinprint receiving region comprises a colour-sensitive coating that changes colour in response to the presence of one or more substances, wherein the method comprises using the electromagnetic radiation detector to detect for the presence of colour.

26. The method of claim 21 or any claim dependent upon claim 21 wherein the method comprises comparing the captured image of the skinprint receiving region with entries in a database of captured skinprint images.

27. The method of claim 26 further comprising seeking a match between the captured image of the skinprint receiving region and one of the entries in the database of captured skinprint images in order to provide an indication of identity of the skinprint.

28. The method of any preceding claim wherein the electromagnetic radiation detector comprises a primary electromagnetic radiation detector and a secondary electromagnetic radiation detector and wherein:
a first of the primary and secondary electromagnetic radiation detectors is used to detect electromagnetic radiation transmitted through the waveguide;
a second of the primary and secondary electromagnetic radiation detectors is used to detect electromagnetic radiation reflected by the waveguide interface.

29. The method of any of claims 1 to 28 wherein the apparatus further comprises a secondary electromagnetic radiation detector and wherein the secondary electromagnetic radiation detector is configured to provide a measure of strength of electromagnetic radiation emitted by the primary electromagnetic radiation source.

30. An apparatus for determining presence of a skinprint, the apparatus comprising:
a translucent waveguide comprising a first surface providing a waveguide interface coincident with a skinprint receiving region;
a primary electromagnetic radiation source coupled to the translucent waveguide such that primary electromagnetic radiation emitted by the primary electromagnetic radiation source reaches the waveguide interface at an angle of incidence relative to and on a first side of a normal line that is perpendicular to the waveguide interface so as to enable reflection of the primary electromagnetic radiation at the waveguide interface where the waveguide interfaces with ambient and so as to enable transmission of primary electromagnetic radiation through the waveguide interface where the waveguide interfaces with a skinprint;
an electromagnetic radiation detector configured to produce a detector signal indicative of either:
(c) a first portion of the primary electromagnetic radiation that undergoes total internal reflection at the waveguide interface; or
(d) a second portion of the primary electromagnetic radiation that undergoes transmission through the waveguide interface; and
a processor configured to process the detector signal and use it to output a result indicative of an extent to which primary electromagnetic radiation behaviour is impacted by presence of a skinprint on the skinprint receiving region.

31. The apparatus of claim 30 further comprising one or more grating couplers between the translucent waveguide and the electromagnetic radiation detector.

32. The apparatus of claim 30 or claim 31 wherein the translucent waveguide is configured to propagate total internal reflection within the translucent waveguide such that internally reflected electromagnetic radiation propagates along the translucent waveguide between the first and a second surface that is parallel to the first surface.

33. The apparatus of any of claims 30 to 32 wherein the reference value represents an expected maximum value in the event that either
(a) all the primary electromagnetic radiation undergoes total internal reflection at the waveguide interface; or
(b) all the primary electromagnetic radiation undergoes transmission through the waveguide interface.

34. The apparatus of claim 33 wherein the reference value is stored in memory of the calculation unit.

35. The apparatus of any of claims 30 to 34 wherein the electromagnetic radiation detector comprises a primary electromagnetic radiation detector and a secondary electromagnetic radiation detector, wherein:
a first one of the primary and secondary electromagnetic radiation detectors is configured to detect the first portion of the primary electromagnetic radiation that undergoes total internal reflection at the waveguide interface; and
a second one of the primary and secondary electromagnetic radiation detectors is configured to detect the second portion of the primary electromagnetic radiation that undergoes transmission through the waveguide interface.

36. The apparatus of any of claims 30 to 35 further comprising a secondary electromagnetic radiation source coupled to the translucent waveguide such that secondary electromagnetic radiation emitted by the secondary electromagnetic radiation source reaches the waveguide interface at a secondary angle of incidence whereby the secondary electromagnetic radiation transmits through the waveguide interface.

37. The apparatus of claim 36 wherein at least one of the primary electromagnetic radiation source and the secondary electromagnetic radiation source is configured to provide pulsed electromagnetic radiation having a pulse pattern and the processor is configured to distinguish between the primary electromagnetic radiation and the secondary electromagnetic radiation based by virtue of the pulse pattern.

38. The apparatus of any of claims 30 to 37 wherein the translucent waveguide comprises an integrated reference feature and wherein the electromagnetic radiation detector is configured to detect the integrated reference feature and produce an output indicative thereof.

39. The apparatus of claim 38 wherein the processor is configured to analyse the output indicative of the integrated reference feature and compare it to a control value for assisting in calculating a quality metric for the skinprint.

40. The apparatus of any of claims 30 to 39 wherein the primary electromagnetic radiation source is configured to produce broad spectrum radiation and wherein the calculation unit is configured to compare a spectrum of the electromagnetic radiation detected by the electromagnetic radiation detector with a spectrum of the electromagnetic radiation of the electromagnetic radiation source.

41. The apparatus of any of claims 30 to 40 further comprising a diffuser, such as a holographic diffuser, between the translucent waveguide and the electromagnetic radiation detector.

42. The apparatus of any of claims 30 to 41 wherein the translucent waveguide is replaceable to allow for replacement between uses.

43. The apparatus of any of claims 30 to 42 wherein the primary electromagnetic radiation source is a light source, optionally one of the following: an LED, a filament bulb, a laser, and a fluorescent bulb.

44. The apparatus of any of claims 36 to 43 wherein the secondary primary electromagnetic radiation source is a light source, optionally one of the following: an LED, a filament bulb, a laser, and a fluorescent bulb.

45. The apparatus of any of claims 30 to 44 wherein the primary electromagnetic radiation source is configured either to produce broad spectrum radiation or to produce a single narrow spectrum of radiation.

46. The apparatus of any of claims 36 to 45 wherein the secondary electromagnetic radiation source is configured either to produce broad spectrum radiation or to produce a single narrow spectrum of radiation.

47. The apparatus of any preceding claim wherein the electromagnetic radiation detector comprises one or more of the following: a photodiode; a phototransistor; a CCD sensor; a photomultiplier; and a light dependent resistor.
